(19) United States
(12) Patent Application Publication
(10) Pub. No.: US 2005/0075351 A1
(43) Pub. Date: Apr. 7, 2005

(54) Use

(76) Inventors: Stefan Berg, Sodertalje (SE); Ratan Bhat, Sodertalje (SE); Sven Hellberg, Sodertalje (SE)

Correspondence Address: WHITE & CASE LLP;PATENT DEPARTMENT, 1155 AVENUE OF THE AMERICAS, NEW YORK, NY, 10036, US

(21) Appl. No.: 10/499,685

(22) PCT Filed: Dec. 18, 2002
(86) PCT No.: PCT/SE02/02372

(30) Foreign Application Priority Data
Dec. 20, 2001 (SE) 0104341-3

(51) Int. Cl. A61K 31/517; C07D 43/02

(52) U.S. Cl. 514/266.2; 544/284

(57) ABSTRACT

The present invention relates to a new use of oxindole derivatives of formula I, as a free base or pharmaceutically acceptable salts thereof, in the manufacture of a medicament for the prevention and/or treatment of dementia related diseases, Alzheimer's Disease and conditions associated with glycogen synthase kinase-3. Formula (I) wherein R¹, R², R³, ring Z, m and n are as defined as in claim 1. The present invention further relates to a method of prevention and/or treatment of dementia related diseases, Alzheimer's Disease and conditions associated with glycogen synthase kinase-3, as well as a pharmaceutical composition for said use.

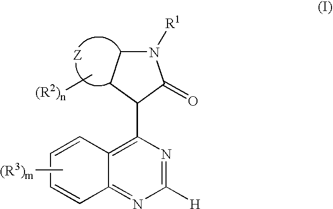

### FIELD OF INVENTION

The present invention relates to a new use of oxindole derivatives, as a free base or pharmaceutically acceptable salts thereof, in the manufacture of a medicament for the treatment and/or prevention of dementia related diseases, Alzheimer's Disease and conditions associated with glycogen synthase kinase-3. The present invention further relates to a method of treatment and/or prevention of dementia related diseases, Alzheimer's Disease and conditions associated with glycogen synthase kinase-3.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on the serine 9 residue and inactivates it.

Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but believed to be related. Glycogen synthase kinase 3β (GSK3β) or Tau (τ) phosphorylating kinase selectively phosphorylates the microtubule associated protein τ in neurons at sites that are hyperphosphorylated in AD brains. Hyperphosphorylated protein τ has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia complex of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of t and a paired helical filaments-like state via induction of GSK3β activity, followed by disruption of axonal transport and neuronal death (Imahori and Uchida., J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

Chronic and Acute Neurodegenerative Diseases.

Growth factor mediated activation of the PI3K/Akt pathway has been shown to play a key role in neuronal survival. The activation of this pathway results in GSK3, inhibition. Recent studies (Bhat et. al., PNAS 97:11074-11079 (2000)) indicate that GSK3, activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK30 may therefore be of therapeutic relevance in the treatment of BD as well as in AD patients that have affective disorders.

Schizophrenia

GSK3 is involved in signal transduction cascades of multiple cellular processes, particularly during neural development. Kozlovsky et al (Am J Psychiatry 2000 May;157(5):831-3) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February;49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

Hair Loss

GSK3 phosphorylates and degrades β-catenin. O-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25;95 (5):605-14)). The new follicles formed sebaceous glands and dermal papilla, normally established only in embryogenesis. Thus GSK3 inhibition may offer treatment for baldness.

Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### DETAILED DESCRIPTION OF THE INVENTION

Compounds of general formula I are disclosed in WO 99/10349. The effect of the compounds on reducing antiangiogenic and/or vascular permeability in mammals has been investigated.

It has now suprisingly been found that the group of oxindole derivatives as decribed in WO 99/10349 are well suited for inhibiting glycogen synthase kinase-3. Said glycogen synthase kinase-3 inhibitors are suitable in the prevention and/or treatment of conditions associated with glycogen synthase kinase-3 in the central and peripheral nervous system. In particular, the compounds of the invention are expected to be suitable for prevention and/or treatment of especially dementia related diseases and Alzheimer's Disease.

The dementia related diseases are selected from the group consisting of Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica. The compounds of the invention are also expected to be suitable for prevention and/or treatment of amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

The compounds of the invention are further expected to be suitable for prevention and/or treatment of Mild Cognitive Impairment, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment and androgenetic alopecia.

In the present invention GSK3 inhibitors of general formula I may be used in the manufacturing of a medicament for the treatment and/or prevention of conditions associated with glycogen synthase kinase-3: 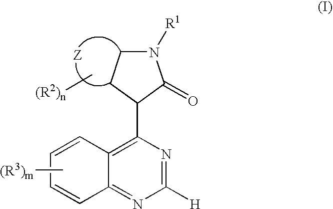
wherein:
ring Z is a 5 or 6 membered heterocyclic ring containing 1 to 3 heteroatoms selected independently from O, N and S but not more than 2 nitrogen atom;
R¹ is hydrogen or C₁₋₃alkyl;
R² is hydroxy, halogeno, fluoromethyl, difluoromethyl, trifluoromethyl, fluoromethoxy, difluoromethoxy, trifluoromethoxy, 2,2,2-trifluoroethyl, cyano, amino, nitro, C₁₋₃alkyl, C₁₋₃alkoxy, C₁₋₃alkanoyloxy, C₂₋₄alkanoyl, C₁₋₄alkanoylamino, C₁₋₄alkoxycarbonyl, C₁₋₄alkylthio, C₁₋₄alkylsulphinyl, C₁₋₄alkylsulphonyl, carbamoyl, N—C₁₋₄alkylcarbamoyl, N,N-di(C₄alkyl)carbamoyl, aminosulphonyl, N—C₁₋₄alkylaminosulphonyl, N,N-di(C₁₋₄alkyl)aminosulphonyl or C₁₋₄alkylsulphonylamino, or
R² is selected from one of the following groups:
   1) R⁴X¹, wherein X¹ is a direct bond, O, NR⁵, C₁₋₃alkyl, C₂₋₄alkanoyl, CONR⁶R⁷, SO₂NR⁸R⁹ or SO₂R¹⁰ (wherein R⁵, R⁶ and R⁸ each independently represent hydrogen or C₁₋₂alkyl and R⁷, R⁹ and R¹⁰ each independently represent C₁₋₄alkyl and wherein R⁴ is linked to R⁷, R⁹ or R¹⁰); and
   R⁴ is phenyl or a 5 or 6 membered heterocyclic group with one or two heteroatoms, selected independently from O, S and N, which heterocyclic group may be saturated or unsaturated and which phenyl or heterocyclic group may be substituted with one or two substituents selected independently from oxo, hydroxy, halogeno, C₁₋₃alkyl, C₁₋₃alkoxy, C₁₋₃alkanoyloxy, trifluoromethyl, cyano, amino, nitro and C₁₋₄alkoxycarbonyl;
   2) X²C₂₋₄alkylX³C₁₋₃alkyl (wherein X² is O or NR¹¹ (wherein R¹¹ is hydrogen, C₁₋₃alkyl or C₁₋₃alkoxyC₂₋₃alkyl) and X³ is O, NR¹², S, SO or SO₂ (wherein R¹² is hydrogen, C₁₋₃alkyl or C₁₋₃alkoxyC₂₋₃alkyl));
   3) C₁₋₂alkylX⁴C₂₋₃alkylX⁵C₁₋₃alkyl (wherein X⁴ and X⁵ each independently represent O, S, SO, SO₂ or NR¹³ (wherein R¹³ is hydrogen, C₁₋₃alkyl or C₁₋₃alkoxyC₂₋₃alkyl)); and
   4) C₁₋₃alkylX⁶C₁₋₃alkyl (wherein X⁶ is O, S, SO, SO₂ or NR¹⁴ (wherein R¹⁴ is hydrogen, C₁₋₃alkyl or C₁₋₃alkoxyC₂₋₃alkyl));
R³ is hydroxy, halogeno, nitro, fluoromethyl, difluoromethyl, trifluoromethyl, fluoromethoxy, difluoromethoxy, trifluoromethoxy, C₁₋₃alkyl, cyano, amino or R¹⁵X⁷,
   wherein X⁷ is a direct bond, O, CH₂, S, SO, SO₂, NR¹⁶CO, CONR¹⁷, SO₂NR¹⁸, NR¹⁹SO₂ or NR²⁰ (wherein R¹⁶, R¹⁷, R¹⁸, R¹⁹ and R²⁰ each independently represent hydrogen, C₁₋₃alkyl or C₁₋₃alkoxyC₂₋₃alkyl); and
   R¹⁵ is selected from one of the following groups:
   1) hydrogen or C₁₋₅alkyl, which may be substituted with one or more groups selected independently from hydroxy, fluoro and amino;
   2) C₁₋₅alkylX⁸COR²¹ (wherein X⁸ is O or NR²² (wherein R²² is hydrogen, C₁₋₃alkyl or C₁₋₃alkoxyC₂₋₃alkyl) and R²¹ is C₁₋₃alkyl, NR²³R²⁴ or OR²⁵ (wherein R²³, R²⁴ and R²⁵ each independently represent hydrogen, C₁₋₃alkyl or C₁₋₃alkoxyC₂₋₃alkyl));
   3) C₁₋₅alkylX⁹R²⁶ (wherein X⁹ is O, S, SO, SO₂, OCO, NR²⁷CO, CONR²⁸, SO₂NR²⁹, NR³⁰SO₂ or NR³¹ (wherein R²⁷, R²⁸, R²⁹, R³⁰ and R³¹ each independently represent hydrogen, C₁₋₃alkyl or C₁₋₃alkoxyC₂₋₃alkyl) and R²⁶ is hydrogen, C₁₋₃alkyl, cyclopentyl, cyclohexyl or a 5 or 6 membered saturated heterocyclic group with one or two heteroatoms selected independently from O, S and N, which C₁₋₃alkyl group may be substituted with one or two substituents selected independently from oxo, hydroxy, halogeno and C₁₋₄alkoxy and which heterocyclic group may be substituted with one or two substituents selected independently from oxo, hydroxy, halogeno, C₁₋₄alkyl,
   C₁₋₄hydroxyalkyl and C₁₋₄alkoxy);
   4) C₁₋₅alkylX¹⁰C₁₋₅alkylX¹¹R³² (wherein X¹⁰ and X¹¹ each independently represent O, S, SO, SO₂, NR³³CO, CONR³⁴, SO₂NR³⁵, NR³⁶SO₂ or NR³⁷ (wherein R³³, R³⁴, R³⁵, R³⁶ and R³⁷ each independently represent hydrogen, C₁₋₃alkyl or C₁₋₃alkoxyC₂₋₃alkyl) and R³² is hydrogen or C₁₋₃alkyl);
   5) R³⁸ (wherein R³⁸ is a 5 or 6 membered saturated heterocyclic group with one or two heteroatoms selected independently from O, S and N, which heterocyclic group may be substituted with one or two substituents selected independently from oxo, hydroxy, halogeno, C₁₋₄alkyl, C₁₋₄hydroxyalkyl and C₁₋₄alkoxy);
   6) C₁₋₅alkylR³⁸ (wherein R³⁸ is as defined hereinbefore),
   7) C₂₋₅alkenylR³⁸ (wherein R³⁸ is as defined hereinbefore);
   8) C₂₋₅alkenylR³⁸ (wherein R³⁸ is as defined hereinbefore);
   9) R³⁹ (wherein R³⁹ is a pyridone group, a phenyl group or a 5 or 6 membered aromatic heterocyclic group with 1 to 3 heteroatoms selected independently from O, N and S, which pyridone, phenyl or heterocyclic group may carry up to 5 substituents selected independently from hydroxy halogeno, amino, C₁₋₄alkyl, C₁₋₄alkoxy, C₁₋₄hydroxyalkyl, C₁₋₄aminoalkyl, C₁₋₄alkylamino, C₁₋₄hydroxyalkoxy, carboxy, trifluoromethyl, cyano, CONR⁴⁰R⁴¹ and NR⁴²COR⁴³ (wherein R⁴⁰, R⁴¹, R⁴² and R⁴³ each independently represent hydrogen, C₁₋₄alkyl or C₁₋₃alkoxyC₂₋₃alkyl));
   10) C₁₋₅alkylR³⁹ (wherein R³⁹ is as defined hereinbefore);
   11) C₂₋₅alkenylR³⁹ (wherein R³⁹ is as defined hereinbefore);
   12) C₂₋₅alkynylR³⁹ (wherein R³⁹ is as defined hereinbefore);
   13) C₁₋₅alkylX¹²R³⁹ (wherein X¹² is O, S, SO, SO₂, NR⁴⁴CO, CONR⁴⁵, SO₂NR⁴⁶, NR⁴⁷SO₂ or NR⁴⁸ (wherein R⁴⁴, R⁴⁵, R⁴⁶, R⁴⁷ and R⁴⁸ each independently represent hydrogen, C₁₋₃alkyl or C₁₋₃alkoxyC₂₋₃alkyl) and R³⁹ is as defined hereinbefore);
   14) C₂₋₅alkenylX¹³R³⁹ (wherein X¹³ is O, S, SO, SO₂, NR⁴⁹CO, CONR⁵⁰, SO₂NR⁵¹, NR⁵²SO₂ or NR⁵³ (wherein R⁴⁹, R⁵⁰, R⁵¹, R⁵² and R⁵³ each independently represent hydrogen, C₁₋₃alkyl or C₁₋₃alkoxyC₂₋₃alkyl) and R³⁹ is as defined hereinbefore);
   15) C₂₋₅alkynylX¹⁴R³⁹ (wherein X¹⁴ is O, S, SO, SO₂, NR⁵⁴CO, CONR⁵⁵, SO₂NR⁵⁶, NR⁵⁷SO₂ or NR⁵⁸ (wherein R⁵⁴, R⁵⁵, R⁵⁶, R⁵⁷ and R⁵⁸ each independently represent hydrogen, C₁₋₃alkyl or C₁₋₃alkoxyC₂₋₃alkyl) and R³⁹ is as defined hereinbefore);
   16) C₁₋₃alkylX¹⁵C₁₋₃alkylR³⁹ (wherein X¹⁵ is O, S, SO, SO₂, NR⁵⁹CO, CONR⁶⁰, SO₂NR⁶¹, NR⁶²SO₂ or NR⁶ (wherein R⁵⁹, R⁶⁰, R⁶¹, R⁶² and R⁶³ each independently represent hydrogen, C₁₋₃alkyl or C₁₋₃alkoxyC₂₋₃alkyl) and R³⁹ is as defined hereinbefore); and
   17) C₁₋₃alkylX¹⁵C₁₋₃alkylR³⁸ (wherein X¹⁵ and R³⁸ are as defined hereinbefore);
n is 0, 1, 2 or 3 when Z is a 6 membered heterocyclic ring and n is 0, 1 or 2 when Z is a membered heterocyclic ring;
m is 0, 1, 2, 3 or 4;
   as a free base or pharmaceutically acceptable salts thereof.

According to one aspect of the present invention compounds of formula I may be used,
wherein R¹, R², R³, m and n are as defined hereinbefore; and
ring Z is a 6 membered heterocyclic ring containing 1 or 2 nitrogen atoms.

In another aspect of the invention compounds of formula I may be used, wherein Z is a 6 membered heterocyclic ring containing 1 or 2 nitrogen atoms and R¹ is hydrogen.

In a further aspect of the invention compounds of formula I may be used, wherein R² is halogeno, C₁₋₃alkyl, trifluoromethyl, cyano, carbamoyl, N—C₁₋₄alkylcarbamoyl, aminosulphonyl or a group R⁴X¹,
wherein X¹ is CONR⁶R⁷ (wherein R⁶ is hydrogen or C₁₋₂alkyl and R⁷ is C₁₋₄alkyl and
wherein R⁴ is linked to R⁷); and
   n is 0 or 1.

In yet another aspect of the invention compounds of formula I may be used, wherein R³ is R¹⁵X⁷,
wherein X⁷ is O; and
R¹⁵ is selected from one of the following groups:
1) hydrogen or C₁₋₅alkyl;
3) C₁₋₅alkylX⁹R²⁶ (wherein X⁹ is O (wherein R²⁶ is hydrogen or C₁₋₃alkyl));
4) C₁₋₅alkylX¹⁰C₁₋₅alkylX¹¹R³² (wherein X¹⁰ and X¹¹ are O, and R³² is hydrogen or C₁₋₃alkyl);
6) C₁₋₅alkylR³⁸ (wherein R³⁸ is a 6 membered saturated heterocyclic group with one or two heteroatoms selected independently from O, S and N, which heterocyclic group may be substituted with one or two substituents selected independently from oxo, hydroxy, halogeno, C₁₋₄alkyl, C₁₋₄hydroxyalkyl and C₁₋₄alkoxy);
7) C₂₋₅alkenylR³⁸ (wherein R³⁸ is as defined hereinbefore);
10) C₁₋₅alkylR³⁹ (wherein R³⁹ is a 5 or 6 membered aromatic heterocyclic group with 1 to 3 heteroatoms selected independently from O, N and S, which heterocyclic group may carry up to 4 substituents selected independently from hydroxy halogeno, amino, C₁₋₄alkyl, C₁₋₄alkoxy, C₁₋₄hydroxyalkyl, C₁₋₄aminoalkyl, C₁₋₄alkylamino, C₁₋₄hydroxyalkoxy, carboxy, trifluoromethyl, cyano, CONR⁴⁰R⁴¹ and NR⁴²COR⁴³ (wherein R⁴⁰, R⁴¹R⁴² and R⁴³ each independently represent hydrogen, C₁₋₄alkyl or C₁₋₃alkoxyC₂₋₃alkyl));
13) C₁₋₅alkylX¹²R³⁹ (wherein X¹² is O and R³⁹ is as defined hereinbefore);
   m is 0, 1 or 2.

One aspect of the invention relates to the use of compounds of formula I, wherein R¹ is hydrogen, Z is a 6 membered heterocyclic ring containing 1 or 2 nitrogen atoms, R² is halogeno or C₁₋₃alkyl and n is 0 or 1, R³ is morpholinopropoxy, dioxothiomorpholino-propoxy, morpholinobutenyl-oxy, pyridyloxy-ethoxy, triazolyl-ethoxy, imidazolyl-ethoxy, methoxy, methoxyethoxy or methoxyethoxy-ethoxy and m is 0, 1, or 2.

In another aspect of the invention, use is made of the following compounds in the manufacturing of a medicament for the treatment and/or prevention of conditions associated with glycogen synthase kinase-3;
4-(7-Azaoxindol-3-yl)-6-methoxy-7-(2-methoxyethoxy)quinazoline,
4-(7-Azaoxindol-3-yl)-6-methoxy-7-(3-morpholinopropoxy)quinazoline,
4-(7-Azaoxindol-3-yl)-7-(2-(imidazol-1-yl)ethoxy)-6-methoxyquinazoline,
4-(5,7-Diaza-6-methyloxindol-3-yl)-7-(3-morpholinopropoxy)quinazoline,
4-(7-Aza-6-chlorooxindol-3-yl)-7-(3-morpholinopropoxy)quinazoline,
4-(5,7-Diaza-6-methyloxindol-3-yl)-6-methoxy-7-(2-(1,2,3-triazol-1-yl)ethoxy)quinazoline,
4-(5,7-Diaza-6-methyloxidol-3-yl)-7-(2-(2-methoxyethoxy)ethoxy)quinazoline,
4-(7-Azaoxindol-3-yl)-7-(3-morpholinopropoxy)quinazoline,
4-(7-Azaoxindol-3-yl)-7-(2-(2-methoxyethoxy)ethoxy)quinazoline,
4-(7-Azaoxindol-3-yl)-7-(4-morpholinobut-2-en-1-yloxy)quinazoline,
4-(5,7-Diaza-6-methyloxindol-3-yl)-6-methoxy-7-(2-(4-pyridyloxy)ethoxy)quinazoline,
4-(7-Aza-6-chlorooxindol-3-yl)-7-(2-(2-methoxyethoxy)ethoxy)quinazoline, and
4-(5,7-Diaza-6-methyloxindol-3-yl)-7-(3-(1,1-dioxothiomorpholino)-propoxy)quinazoline;
   as a free base or pharmaceutically acceptable salts thereof.

For the avoidance of doubt it is to be understood that where in this specification a group is qualified by ‘hereinbefore defined’ or ‘defined hereinbefore’ the said group encompasses the first occurring and broadest definition as well as each and all of the preferred definitions of that group.

For the avoidance of doubt it is to be understood that in this specification ‘C₁₋₅’ means a carbon group having 1, 2, 3, 4 or 5 carbon atoms.

In this specification, unless stated otherwise, the term “alkyl” includes both straight and branched chain alkyl groups. C₁₋₅alkyl may be methyl, ethyl, n-propyl, i-propyl, n-butyl, i-butyl, s-butyl, t-butyl, n-pentyl, i-pentyl, t-pentyl, neo-pentyl.

The term “alkoxy” as used herein, unless stated otherwise includes “alkyl” O groups in which “alkyl” is as hereinbefore defined. C₁₋₅alkoxy may be methoxy, ethoxy, n-propoxy, i-propoxy, n-butoxy, i-butoxy, s-butoxy, t-butoxy, n-pentyloxy, i-pentyloxy, t-pentyloxy, neo-pentyloxy.

The term “alkanoyl” as used herein, unless otherwise stated includes formyl and alkylC═O groups in which “alkyl” is as defined hereinbefore, for example C₂alkanoyl is ethanoyl and refers to CH₃C═O, C₁alkanoyl is formyl and refers to CHO.

In this specification, unless stated otherwise, the term “alkenyl” includes both straight and branched chain alkenyl groups but references to individual alkenyl groups such as 2-butenyl are specific for the straight chain version only. Unless otherwise stated, the term “alkenyl” advantageously refers to chains with 2 to 5 carbon atoms, preferably 3 to 4 carbon atoms.

In this specification, unless stated otherwise, the term “alkynyl” includes both straight and branched chain alkynyl groups but references to individual alkynyl groups such as 2-butynyl are specific for the straight chain version only. Unless otherwise stated, the term “alkynyl” advantageously refers to chains with 2 to carbon atoms, preferably 3 to 4 carbon atoms.

In this specification, unless stated otherwise, the term “5 or 6 membered heterocyclic group with one or two heteroatoms, selected independently from O, S and N, which heterocyclic group may be saturated or unsaturated” or “5 or 6 membered heterocyclic ring containing 1 to 3 heteroatoms selected independently from O, N and S, which heterocyclic group may be saturated or unsaturated”, includes both heteroaromatic rings and heterocyclic rings that are saturated. Examples of such heterocyclic groups includes, but are not limited to furyl, imidazolyl, isoxazolyl, isothiazolyl, oxazolyl, pyrazinyl, pyrazolyl, pyridazinyl, pyridyl, pyrimidyl, pyrrolyl, thiazolyl, thienyl, imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl or thiomorpholinyl.

In this specification, unless stated otherwise, the term “5 or 6 membered saturated heterocyclic group with one or two heteroatoms, selected independently from O, S and N” may be, but are not limited to imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl or thiomorpholinyl.

In this specification, unless stated otherwise, the term “5 or 6 membered aromatic heterocyclic group with 1 to 3 heteroatoms, selected independently from O, N and S” may be, but are not limited to furyl, imidazolyl, isoxazolyl, isothiazolyl, oxazolyl, pyrazinyl, triazinyl, pyrazolyl, pyridazinyl, pyridyl, pyrimidyl, pyrrolyl, thiazolyl or thienyl.

In this specification, unless stated otherwise, the term “5 or 6 membered heterocyclic ring containing 1 to 3 heteroatoms selected independently from O, N and S” may be, but are not limited to furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyi.

In this specification, unless stated otherwise, the term halogeno may be fluor, chlorine, bromine or iodine.

For the avoidance of any doubt, it is to be understood that when X⁷ is, for example, a group of formula NR¹⁶CO, it is the nitrogen atom be substituted withing the R¹⁶ group which is attached to the quinazoline ring and the carbonyl (CO) group is attached to R¹⁵, whereas when X⁷ is, for example, a group of formula CONR¹⁷, it is the carbonyl group which is attached to the quinazoline ring and the nitrogen atom be substituted withing the R¹⁷ group is attached to R¹⁵. A similar convention applies to the other two atoms X⁷ linking groups such as NR¹⁹SO₂ and SO₂NR¹⁸. When X⁷ is NR²⁰ it is the nitrogen atom be substituted withing the R²⁰ group, which is linked to the quinazoline ring and to R¹⁵. An analogous convention applies to other groups. It is further to be understood that when X⁷ represents NR²⁰ and R²⁰ is C₁₋₃alkoxyC₂₋₃alkyl it is the C₂₋₃alkyl moiety, which is linked to the nitrogen atom of X⁷ and an analogous convention applies to other groups.

For the avoidance of any doubt, it is to be understood that in a compound of formula I when R¹⁵ is, for example, a group of formula C₁₋₅alkylX¹⁵C₁₋₅alkylR³⁹, it is the terminal C₁₋₅alkyl moiety, which is linked to X¹⁵, similarly when R¹⁵ is, for example, a group of formula C₂₋₅alkenylR³⁹ it is the C₂₋₅alkenyl moiety, which is linked to X⁷ and an analogous convention applies to other groups.

For the avoidance of any doubt, it is to be understood that when R³⁹ carries a C₁₋₄aminoalkyl substituent it is the C₁₋₄alkyl moiety, which is attached to R³⁹ whereas when R³⁹ carries a C₁₋₄alkylamino substituent it is the amino moiety, which is attached to R³⁹ and an analogous convention applies to other groups.

For the avoidance of any doubt when X¹ is C₂₋₄alkanoyl it is the carbonyl moiety, which is linked to the heteroaromatic oxindole group and it is the alkyl moiety, which is linked to R⁴ and an analogous convention applies to other groups.

For the avoidance of any doubt when R² is a group X²C₂₋₄alkylX³C₁₋₃alkyl it is X², which is linked to the heteroaromatic oxindole group and an analogous convention applies to other groups. When R² is a group C₁₋₂alkylX⁴C₂₋₃alkylX⁵C₁₋₃alkyl it is the C₁₋₂alkyl moiety, which is linked to the heteroaromatic oxindole group and an analogous convention applies to other groups.

Some compounds of formula I may have chiral centres and/or geometric isomeric centres (E- and Z-isomers), and it is to be understood that the invention encompasses all such optical, diastereoisomers and geometric isomers that possess GSK3 inhibitory activity.

It is to be understood that the present invention also relates to any and all tautomeric forms of the compounds of formula I.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

Both organic and inorganic acids can be employed to form non-toxic pharmaceutically acceptable acid addition salts of the compounds of this invention. In addition, a suitable pharmaceutically acceptable salt of the compounds of the invention is an alkali metal salt, an alkaline earth metal salt or a salt with an organic base.

Compound of formula I, or salt thereof, may be prepared by any process known to be applicable to the preparation of chemically-related compounds. Such processes include, for example, those illustrated in European Patent Applications Publication Nos. 0520722, 0566226, 0602851, 0635498 and 0636608 and PCT application WO 99/10349.

Pharmaceutical Composition

According to one aspect of the present invention there is provided a pharmaceutical composition comprising a compound of formula I, as a free base or salts thereof, for use in prevention and/or treatment of dementia related diseases, Alzheimer's Disease and conditions associated with glycogen synthase kinase-3 and other conditions listed below.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream or for rectal administration as a suppository.

In general the above compositions may be prepared in a conventional manner using pharmaceutically acceptable carriers or diluents.

Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

Illustrate representative pharmaceutical dosage forms containing a compound of formula I, as a free base or salts thereof, are described in WO 99/10349.

Medical Use

Surprisingly, it has been found that the compounds defined in the present invention, as a free base or salts thereof, are useful in therapy. The compounds of the present invention are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable in the manufacture of a medicament for the prevention and/or treatment of dementia related diseases and Alzheimer's Disease.

The dementia related diseases are selected from the group consisting of Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica. The compounds of the invention are also expected to be suitable in the manufacture of a medicament for the prevention and/or treatment of amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

The compounds of the invention are further expected to be suitable in the manufacture of a medicament for the prevention and/or treatment of Mild Cognitive Impairment, Age-Associated Memory Impairment, Age-Related Cognitive Decline, Cognitive Impairement No Dementia, mild cognitive decline, mild neurocognitive decline, Late-Life Forgetfulness, memory impairment and cognitive impairment and androgenetic alopecia.

The present invention relates also to the use of a compound of formula I as defined hereinbefore, in the manufacture of a medicament for the prevention and/or treatment of conditions associated with glycogen synthase kinase-3.

The invention also provides for a method of prevention and/or treatment of dementia related diseases, Alzheimer's Disease and conditions associated with glycogen synthase kinase-3 and other conditions listed above comprising administrering to a mammal, including man, in need of such prevention and/or treatment a therapeutically effective amount of a compound of formula I, as hereinbefore defined.

In the context of the present specification, the term “therapy” also includes “prevention” unless there are specific indications to the contrary. The terms “therapeutic” and “therapeutically” should be construed accordingly.

Non-Medical Use

In addition to their use in therapeutic medicine, the compounds of formula I as a free base or salts thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

Pharmacology

Determination of ATP Competition in Scintillation Proximity GSK3β Assay.

GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 Ci [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:
ATP Adenosine Triphophatase
BSA Bovin Serum Albumin
EDTA Ethylenediaminetetraacetic acid
GSK3 Glycogen synthase kinase 3
MOPS Morpholinepropanesulfonic acid
SPA Scintillation Proximity Assay.
   Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

1. A method for the treatment and/or prevention of conditions associated with glycogen synthase kinase-3 which comprises administering to a patient in need of such treatment and/or prevention a therapeutically effective amount of a compound of formula I, 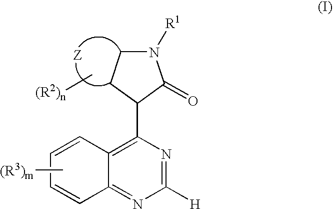
wherein the compound of formula I is a free base or pharmaceutically acceptable salt thereof;
wherein:
ring Z is a 5 or 6 membered heterocyclic ring containing 1 to 3 heteroatoms selected independently from O, N and S but not more than 2 nitrogen atoms;
R¹ is hydrogen or C₁₋₃alkyl;
R² is hydroxy, halogeno, fluoromethyl, difluoromethyl, trifluoromethyl, fluoromethoxy, difluoromethoxy, trifluoromethoxy, 2,2,2-trifluoroethyl, cyano, amino, nitro, C₁₋₃alkyl, C₁₋₃alkoxy, C₁₋₃alkanoyloxy, C₂₋₄alkanoyl, C₁₋₄alkanoylamino, C₁₋₄alkoxycarbonyl, C₁₋₄alkylthio, C₁₋₄alkylsulphinyl, C₁₋₄alkylsulphonyl, carbamoyl, N—C₁₋₄alkylcarbamoyl, N,N-di(C₁₋₄alkyl)carbamoyl, aminosulphonyl, N—C₁₋₄alkylaminosulphonyl, N,N-di(C₁₋₄alkyl)aminosulphonyl or C₁₋₄alkylsulphonylamino, or
R² is selected from one of the following groups:
1) R⁴X¹, wherein X¹ is a direct bond, O, NR⁵, C₁₋₃alkyl, C₂₋₄alkanoyl, CONR⁶R⁷, SO₂NR⁸R⁹ or SO₂R¹⁰ (wherein R⁵, R⁶ and R⁸ each independently represent hydrogen or C₁₋₂alkyl and R⁷, R⁹ and R¹⁰ each independently represent C₁₋₄alkyl and wherein R⁴ is linked to R⁷, R⁹ or R¹⁰); and
R⁴ is phenyl or a 5 or 6 membered heterocyclic group with one or two heteroatoms, selected independently from O, S and N, which heterocyclic group may be saturated or unsaturated and which phenyl or heterocyclic group may be substituted with one or two substituents selected independently from oxo, hydroxy, halogeno, C₁₋₃alkyl, C₁₋₃alkoxy, C₁₋₃alkanoyloxy, trifluoromethyl, cyano, amino, nitro and C₁₋₄alkoxycarbonyl;
2) X²C₂₋₄alkylX³C₁₋₃alkyl (wherein X² is O or NR¹¹ (wherein R¹¹ is hydrogen, C₁₋₃alkyl or C₁₋₃alkoxyC₂₋₃alkyl) and X³ is O, NR¹², S, SO or SO₂ (wherein R¹² is hydrogen, C₁₋₃alkyl or C₁₋₃alkoxyC₂₋₃alkyl));
3) C₁₋₂alkylX⁴C₂₋₃alkylX⁵C₁₋₃alkyl (wherein X⁴ and X⁵ each independently represent O, S, SO, SO₂ or NR¹³ (wherein R¹³ is hydrogen, C₁₋₃alkyl or C₁₋₃alkoxyC₂₋₃alkyl)); and
4) C₁₋₃alkylX⁶C₁₋₃alkyl (wherein X⁶ is O, S, SO, SO₂ or NR¹⁴ (wherein R¹⁴ is hydrogen, C₁₋₃alkyl or C₁₋₃alkoxyC₂₋₃alkyl));
R³ is hydroxy, halogeno, nitro, fluoromethyl, difluoromethyl, trifluoromethyl, fluoromethoxy, difluoromethoxy, trifluoromethoxy, C₁₋₃alkyl, cyano, amino or R¹⁵X⁷,
wherein X⁷ is a direct bond, O, CH₂, S, SO, SO₂, NR¹⁶CO, CONR¹⁷, SO₂NR¹⁸, NR¹⁹SO₂ or NR²⁰ (wherein R¹⁶, R¹⁷, R¹⁸, R¹⁹ and R²⁰ each independently represent hydrogen, C₁₋₃alkyl or C₁₋₃alkoxyC₂₋₃alkyl); and
R¹⁵ is selected from one of the following groups:
1) hydrogen or C₁₋₅alkyl, which may be substituted with one or more groups selected independently from hydroxy, fluoro and amino;
2) C₁₋₅alkylX⁸COR²¹ (wherein X⁸ is O or NR²² (wherein R²² is hydrogen, C₁₋₃alkyl or C₁₋₃alkoxyC₂₋₃alkyl) and R²¹ is C₁₋₃alkyl, NR²³R²⁴ or OR²⁵ (wherein R²³, R²⁴ and R²⁵ each independently represent hydrogen, C₁₋₃alkyl or C₁₋₃alkoxyC₂₋₃alkyl));
3) C₁₋₅alkylX⁹R²⁶ (wherein X⁹ is O, S, SO, SO₂, OCO, NR²⁷CO, CONR²⁸, SO₂NR²⁹, NR³⁰ SO₂ or NR³¹ (wherein R²⁷, R²⁸, R²⁹, R³⁰ and R³¹ each independently represent hydrogen, C₁₋₃alkyl or C₁₋₃alkoxyC₂₋₃alkyl) and R²⁶ is hydrogen, C₁₋₃alkyl, cyclopentyl, cyclohexyl or a 5 or 6 membered saturated heterocyclic group with one or two heteroatoms selected independently from O, S and N, which C₁₋₃alkyl group may be substituted with one or two substituents selected independently from oxo, hydroxy, halogeno and C₁₋₄alkoxy and which heterocyclic group may be substituted with one or two substituents selected independently from oxo, hydroxy, halogeno, C₁₋₄alkyl, C₁₋₄hydroxyalkyl and C₁₋₄alkoxy);
4) C₁₋₅alkylX¹⁰C₁₋₅alkylX¹¹R³² (wherein X¹⁰ and X¹¹ each independently represent O, S, SO, SO₂, NR³³CO, CONR³⁴, SO₂NR³⁵, NR³⁶SO₂ or NR³⁷ (wherein R³³, R³⁴, R³⁵, R³⁶ and R³⁷ each independently represent hydrogen, C₁₋₃alkyl or C₁₋₃alkoxyC₂₋₃alkyl) and R³² is hydrogen or C₁₋₃alkyl);
5) R³⁸ (wherein R³⁸ is a 5 or 6 membered saturated heterocyclic group with one or two heteroatoms selected independently from O, S and N, which heterocyclic group may be substituted with one or two substituents selected independently from oxo, hydroxy, halogeno, C₁₋₄alkyl, C₁₋₄hydroxyalkyl and C₁₋₄alkoxy);
6) C₁₋₅alkylR³⁸ (wherein R³⁸ is as defined hereinbefore);
7) C₂₋₅alkenylR³⁸ (wherein R³⁸ is as defined hereinbefore);
8) C₂₋₅alkynylR³⁸ (wherein R³⁸ is as defined hereinbefore);
9) R³⁹ (wherein R³⁹ is a pyridone group, a phenyl group or a 5 or 6 membered aromatic heterocyclic group with 1 to 3 heteroatoms selected independently from O, N and S, which pyridone, phenyl or heterocyclic group may carry up to 5 substituents selected independently from hydroxy halogeno, amino, C₁₋₄alkyl, C₁₋₄alkoxy, C₁₋₄hydroxyalkyl, C₁₋₄aminoalkyl, C₁₋₄alkylamino, C₁₋₄hydroxyalkoxy, carboxy, trifluoromethyl, cyano, CONR⁴⁰R⁴¹ and NR⁴² COR⁴³ (wherein R⁴⁰, R⁴¹, R⁴² and R⁴³ each independently represent hydrogen, C₁₋₄alkyl or C₁₋₃alkoxyC₂₋₃alkyl));
10) C₁₋₅alkylR³⁹ (wherein R³⁹ is as defined hereinbefore);
11) C₂₋₅alkenylR³⁹ (wherein R³⁹ is as defined hereinbefore);
12) C₂₋₅alkynylR³⁹ (wherein R³⁹ is as defined hereinbefore);
13) C₁₋₅alkylX¹²R³⁹ (wherein X¹² is O, S, SO, SO₂, NR⁴⁴CO, CONR⁴⁵, SO₂NR⁴⁶, NR⁴⁷SO₂ or NR⁴⁸ (wherein R⁴⁴, R⁴⁵, R⁴⁶, R⁴⁷ and R⁴⁸ each independently represent hydrogen, C₁₋₃alkyl or C₁₋₃alkoxyC₂₋₃alkyl) and R³⁹ is as defined hereinbefore);
14) C₂₋₅alkenylX¹³R³⁹ (wherein X¹³ is O, S, SO, SO₂, NR⁴⁹CO, CONR⁵⁰, SO₂NR⁵¹, NR⁵² SO₂ or NR⁵³ (wherein R⁴⁹, R⁵⁰, R⁵¹, R⁵² and R⁵³ each independently represent hydrogen, C₁₋₃alkyl or C₁₋₃alkoxyC₂₋₃alkyl) and R³⁹ is as defined hereinbefore);
15) C₂₋₅alkynylX¹⁴R³⁹ (wherein X¹⁴ is O, S, SO, SO₂, NR⁵⁴CO, CONR⁵⁵, SO₂NR⁵⁶, NR⁵⁷SO₂ or NR⁵⁸ (wherein R⁵⁴, R⁵⁵, R⁵⁶, R⁵⁷ and R⁵⁸ each independently represent hydrogen, C₁₋₃alkyl or C₁₋₃alkoxyC₂₋₃alkyl) and R³⁹ is as defined hereinbefore);
16) C₁₋₃alkylX¹⁵C₁₋₃alkylR³⁹ (wherein X¹⁵ is O, S, SO, SO₂, NR⁵⁹ CO, CONR⁶⁰, SO₂NR⁶¹, NR⁶² SO₂ or NR⁶ (wherein R⁵⁹, R⁶⁰, R⁶¹, R⁶² and R⁶³ each independently represent hydrogen, C₁₋₃alkyl or C₁₋₃alkoxyC₂₋₃alkyl) and R³⁹ is as defined hereinbefore); and
17) C₁₋₃alkylX¹⁵C₁₋₃alkylR³⁸ (wherein X¹⁵ and R³⁸ are as defined hereinbefore);
n is 0, 1, 2 or 3 when Z is a 6 membered heterocyclic ring and n is 0, 1 or 2 when Z is a 5 membered heterocyclic ring;
m is 0, 1, 2, 3 or 4.

2. The method according to claim 1, wherein Z is a 6 membered heterocyclic ring containing 1 or 2 nitrogen atoms and R¹ is hydrogen.

3. The method according to claim 1, wherein R² is halogeno, C₁₋₃alkyl, trifluoromethyl, cyano, carbamoyl, N—C₁₋₄alkylcarbamoyl, aminosulphonyl or a group R⁴X¹,
wherein X¹ is CONR⁶R⁷ (wherein R⁶ is hydrogen or C₁₋₂alkyl and
R⁷ is C₁₋₄alkyl and wherein R⁴ is linked to R⁷); and
n is 0 or 1.

4. The method according to claim 1,
wherein R³ is R¹⁵X⁷,
wherein X⁷ is O; and
R¹⁵ is selected from one of the following groups:
1) hydrogen or C₁₋₅alkyl;
3) C₁₋₅alkylX⁹R²⁶ (wherein X⁹ is O (wherein R²⁶ is hydrogen or C₁₋₃alkyl));
4) C₁₋₅alkylX¹⁰C₁₋₅alkylX¹¹R³² (wherein X¹⁰ and X¹¹ are O, and R³² is hydrogen or C₁₋₃alkyl);
6) C₁₋₅alkylR³⁸ (wherein R³⁸ is a 6 membered saturated heterocyclic group with one or two heteroatoms selected independently from O, S and N, which heterocyclic group may be substituted with one or two substituents selected independently from oxo, hydroxy, halogeno, C₁₋₄alkyl, C₁₋₄hydroxyalkyl and C₁₋₄alkoxy);
7) C₂₋₅alkenylR³⁸ (wherein R³⁸ is as defined hereinbefore);
10) C₁₋₅alkylR³⁹ (wherein R³⁹ is a 5 or 6 membered aromatic heterocyclic group with 1 to 3 heteroatoms selected independently from O, N and S, which heterocyclic group may carry up to 4 substituents selected independently from hydroxy halogeno, amino, C₁₋₄alkyl, C₁₋₄alkoxy, C₁₋₄hydroxyalkyl, C₁₋₄aminoalkyl, C₁₋₄alkylamino, C₁₋₄hydroxyalkoxy, carboxy, trifluoromethyl, cyano, CONR⁴⁰R⁴¹ and NR⁴²COR⁴³ (wherein R⁴⁰, R⁴¹, R⁴² and R⁴³ each independently represent hydrogen, C₁₋₄alkyl or C₁₋₃alkoxyC₂₋₃alkyl));
13) C₁₋₅alkylX¹²R³⁹ (wherein X¹² is O and R³⁹ is as defined hereinbefore);
m is 0, 1 or 2.

5. The method according to claim 1, wherein the compound is selected from the group comprising of:
4-(7-Azaoxindol-3-yl)-6-methoxy-7-(2-methoxyethoxy)quinazoline,
4-(7-Azaoxindol-3-yl)-6-methoxy-7-(morpholinopropoxy)quinazoline,
4-(7-Azaoxindol-3-yl)-7-(2-(imidazol-1-yl)ethoxy)-6-methoxyquinazoline,
4-(5,7-Diaza-6-methyloxindol-3-yl)-7-(3-morpholinopropoxy)quinazoline,
4-(7-Aza-6-chlorooxindol-3-yl)-7-(3-morpholinopropoxy)quinazoline,
4-(5,7-Diaza-6-methyloxindol-3-yl)-6-methoxy-7-(2-(1,2,3-triazol-1-yl)ethoxy)quinazoline,
4-(5,7-Diaza-6-methyloxindol-3-yl)-7-(2-(2-methoxyethoxy)ethoxy)quinazoline,
4-(7-Azaoxindol-3-yl)-7-(3-morpholinopropoxy)quinazoline,
4-(7-Azaoxindol-3-yl)-7-(2-(2-methoxyethoxy)ethoxy)quinazoline,
4-(7-Azaoxindol-3-yl)-7-(4-morpholinobut-2-en-1-yloxy)quinazoline,
4-(5,7-Diaza-6-methyloxindol-3-yl)-6-methoxy-7-(2-(4-pyridyloxy)ethoxy)quinazoline,
4-(7-Aza-6-chlorooxindol-3-yl)-7-(2-(2-methoxyethoxy)ethoxy)quinazoline, and
4-(5,7-Diaza-6-methyloxindol-3-yl)-7-(3-(1,1-dioxothiomorpholino)-propoxy)quinazoline; wherein the compound is a free base or pharmaceutically acceptable salt thereof.

6. The method according to claim 1, wherein the treatment and/or prevention of conditions associated with administering to a patient in need of such prevention and/or treatment comprises at least one of the group consisting of dementia related diseases and Alzheimer's Disease.

7. The method for the prevention and/or treatment according to claim 6, wherein the dementia related diseases are selected from the group consisting of Frontotemporal dementia Parkinson's Type, Parkinson dementia complex of Gaum, HIV dementia, diseases with associated neurofibrillar tangle pathologies, predemented states, vascular dementia, dementia with Lewy bodies, Frontotemporal dementia and dementia pugilistica.

8. (canceled)

9. (canceled)

10. A pharmaceutical composition for use in prevention and/or treatment of dementia related diseases, Alzheimer's Disease and conditions associated with glycogen synthase kinase-3, comprising a therapeutically effective amount of a compound of formula I as defined in any one of claims 1 to 5 and pharmaceutically acceptable carriers or diluents.

11. (canceled)

12. A method of prevention and/or treatment of a medical condition selected from the group consisting of amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication, the method comprising administering to a mammal in need of such prevention and/or treatment, a therapeutically effective amount of the compound of formula I as defined in claim 1.

13. A method of prevention and/or treatment of a medical condition selected from the group consisting of Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory and cognitive impairment and androgenetic alopecia, the method comprising administering to a mammal in need of such prevention and/or treatment, a therapeutically effective amount of the compound of formula I as defined in claim 1.
